(19) United States
(12) Patent Application Publication
(10) Pub. No.: US 2020/0194121 A1
(43) Pub. Date: Jun. 18, 2020

(54) Personalized Digital Health System Using Temporal Models

(71) Applicant: Gali Health, Inc., San Francisco, CA (US)

(72) Inventors: Ilya Kupershmidt, San Francisco, CA (US); Arielle S. Radin, Los Angeles, CA (US)

(21) Appl. No.: 16/785,287

(22) Filed: Feb. 7, 2020

Related U.S. Application Data
(63) Continuation of application No. PCT/US2018/045667, filed on Aug. 7, 2018.
(60) Provisional application No. 62/548,918, filed on Aug. 22, 2017.

(51) Int. Cl. G16H 50/20; G16H 10/60; G16H 80/00; A61B 5/00

(52) U.S. Cl. CPC G16H 50/20; G16H 10/60; A61B 5/7275; A61B 5/6802; G16H 80/00

(57) ABSTRACT

A digital health system provides personalized feedback to users using machine learning models. The system receives event data describing physiological events of a user. The system may classify the physiological events and update a temporal model of physiological health of the user using the classified physiological events. The temporal model may map the classified physiological events to a timeline based on timestamps of the events. The temporal model may identify statistical distributions of the physiological events and determine a physiological condition of the user based on the statistical distributions. In some embodiments, the system communicates with a user using a digital health assistant via chatbot interface. The digital health assistant, may be trained using artificial intelligence of the system and simulates interaction with a doctor or health provider.

### CROSS REFERENCE TO RELATED APPLICATION

This application is a continuation of International Patent Application No. PCTUS2018/045667, filed Aug. 7, 2018 which claims priority to U.S. Provisional Patent Application No. 62/548,918, filed Aug. 22, 2017, which is incorporated by reference in its entirety.

### BACKGROUND

### 1. Field of Art

This disclosure relates to temporal models of physiological events of users and particularly to providing personalized feedback based on the temporal models.

### 2. Description of the Related Art

Healthcare organizations, biomedical research agencies, and the patient community increasingly recognize the need to implement a patient-centered approach to health management. Collaboration between patients, family and friends, physicians, and various health providers may contribute to improving a patient's health conditions. Active patient and doctor participation over the course of a patient's life may be particularly key for chronic diseases. Patients desire more ways to take ownership of their health, by learning about diseases, making decisions on treatments, or managing symptoms and lifestyle choices. However, existing systems may not effectively combine health data from various sources, and thus existing systems do not provide customized feedback for users based on their specific health context.

### SUMMARY

A digital health system provides personalized health feedback to users based on various types of models. The models may define health trajectories of diseases, user behaviors, and patient-specific context. The models may be trained using machine learning techniques and using health data input by users or received from different sources of health providers such as physicians, electronic medical records, or curated literature from medical professionals. In some embodiments, a temporal model maps physiological events experienced by a user to a timeline of the user's health journey. As used herein, a physiological event may refer to any type of event that is associated with a user's physiology, health, or lifestyle, as well as relevant actions performed by a user. For instance, physiological events may be medical (e.g., visiting a doctor, taking medication, determining a diagnosis, etc.) or social (e.g., attending an activity with other people, feeling anti-social, having a certain state of mental health, etc.). Using the temporal model and associations between mapped physiological events, the digital health system may determine the likelihood of certain physiological conditions occurring in response to the user's actions. For instance, taking multiple medications may result in a side effect, depending on the user's particular health condition or other relevant context. In an example use case, users may interact with the digital health system via a digital health assistant “chatbot” that encourages users to actively manage and improve their health. This digital health assistant provides easy access by a user to his/her medical information throughout the user's life such that the user can make informed, personalized decisions, for example, including tracking the user's health over time, learning based the user's information and information from the community of patients and medical professionals, and facilitating the user's interactions with his/her physician by providing a continuous stream of health data for the user.

### BRIEF DESCRIPTION OF DRAWINGS

FIG. 1 is a diagram of a computing environment for providing personalized health feedback by a digital health system according to one embodiment.

FIG. 2 is a block diagram of the digital health system according to one embodiment.

FIG. 3 is a data flow diagram of the digital health system according to one embodiment.

FIG. 4A is a diagram of a personalized temporal model for a user of the digital health system according to one embodiment.

FIG. 4B is a diagram of a timeline of the personalized temporal model illustrating statistical distributions according to one embodiment.

FIG. 5 is a flow chart illustrating a process for providing data describing a physiological condition according to one embodiment.

FIGS. 6A, 6B, 6C, and 6D are diagrams of a user interface for a digital health assistant according to various embodiments.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### I. System Overview

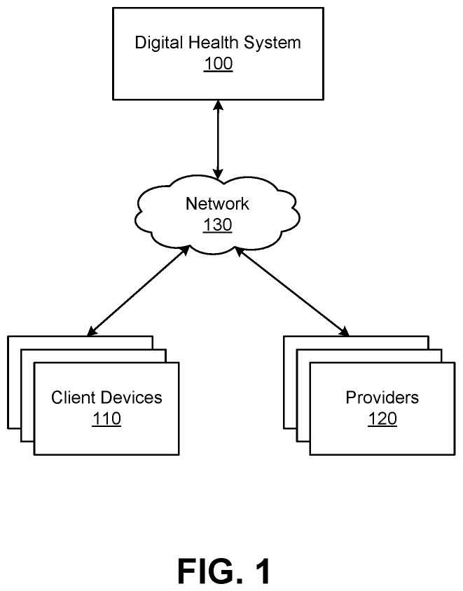

FIG. 1 is a diagram of a computing environment for providing personalized health feedback by a digital health system 100 according to one embodiment. The computing environment includes the digital health system 100, one or more client devices 110, and one or more providers 120 each connected to a network 130. Some embodiments of the computing environment may have additional, fewer, or different components than the ones described herein. The functions can be distributed among the components in a different manner than described in FIG. 1.

A user can interact with the digital health system 100 through the client device 110, e.g., to access health feedback or interact with a digital health assistant “chatbot.” A client device 110 can be a personal or mobile computing device, such as a smartphone, a tablet, or a notebook computer. In some embodiments, the client device 110 executes a client application that uses an application programming interface (API) to communicate with the digital health system 100 through the network 130. The client application of the client device 110 can present information received from the digital health system 100 on a user interface, such as a health recommendations or a timeline of physiological events of a user.

In some embodiments, a client device 110 is a wearable device that captures sensor data of a user. The sensor data may include the physiological data of the user, e.g., heart rate, the blood pressure, calorie and nutrition levels, weight, activity level, rest and sleep levels, brain activity (e.g., electroencephalography data), temperature, among other types of health data and activity information. The wearable device may send sensor data to the digital health system 100, for example, via another client device 110 such as a smartphone.

In some embodiments, providers 120 are computer servers including one or more databases storing health information or physiological data of users. The digital health system 100 may use an API to communicate with providers 120, which may be associated with third party applications. A provider 120 may be associated with the Center for Disease Control and Prevention (CDC) and include health guidelines describing prevention programs for different types of diseases, as well as physiological or psychological conditions. Further, providers 120 may include information from peer review literature and research on various health topics.

In an embodiment, a provider 120 provides electronic medical record (EMR) data. The EMR data includes, for example, medical histories, doctor and hospital visits, medications, allergies, immunizations, medical test results, billing information, demographic data, etc., of the users. The EMR data may be updated over time based on information provided by health care providers such as a user's personal care physician or a nurse, or based on information provided by a user herself or himself.

Client devices 110 and providers 120 can communicate with the digital health system 100 via the network 130, which may comprise any combination of local area and wide area networks employing wired or wireless communication links. In one embodiment, the network 130 uses standard communications technologies and Internet protocols. For example, the network 130 includes communication links using technologies such as the Internet, 3G, 4G, BLUETOOTH®, or WiFi. In some embodiments, all or some of the communication links of the network 130 may be encrypted.

### II. Example Digital Health System

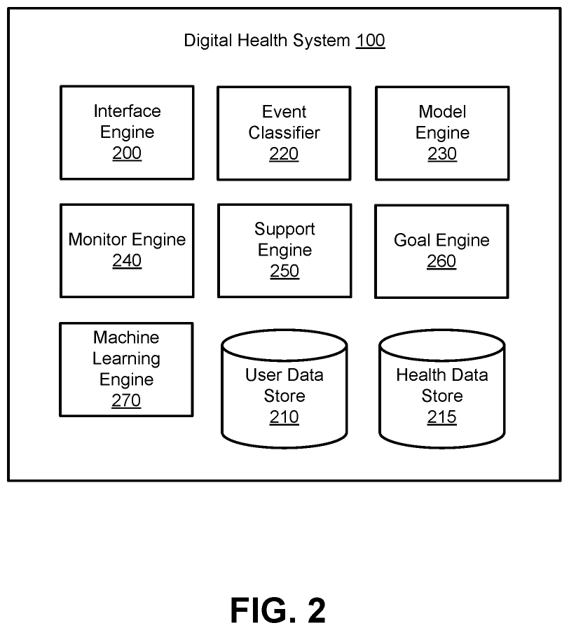

FIG. 2 is a block diagram of the digital health system 100 according to one embodiment. The digital health system 100 includes an interface engine 200, user data store 210, health data store 215, event classifier 220, model engine 230, monitor engine 240, support engine 250, goal engine 260, and machine learning engine 270. In other embodiments, the digital health system 100 may include additional, fewer, and/or different components for various applications.

The interface engine 200 is the interface between users of the digital health system 100, providers 120, and knowledge learned by the digital health system 100. The interface engine 200 receives information about users of the digital health system 100, diseases, and various health information. The interface engine 200 may also provide digital health assistant functionality, which may simulate interaction with a doctor or another type of health provider. In contrast to seeing a doctor in-person, the digital health assistant provides a virtual experience with which users can interact using a client device 120 such as a smartphone. The digital health assistant does not necessarily replace a user's health providers. Rather, the digital health assistant may facilitate interactions between the user and the user's health providers, or aggregate or augment information from the health providers. The digital health assistant may be referred to by a name, for example, “Gali,” which may help engender human interaction or empathy.

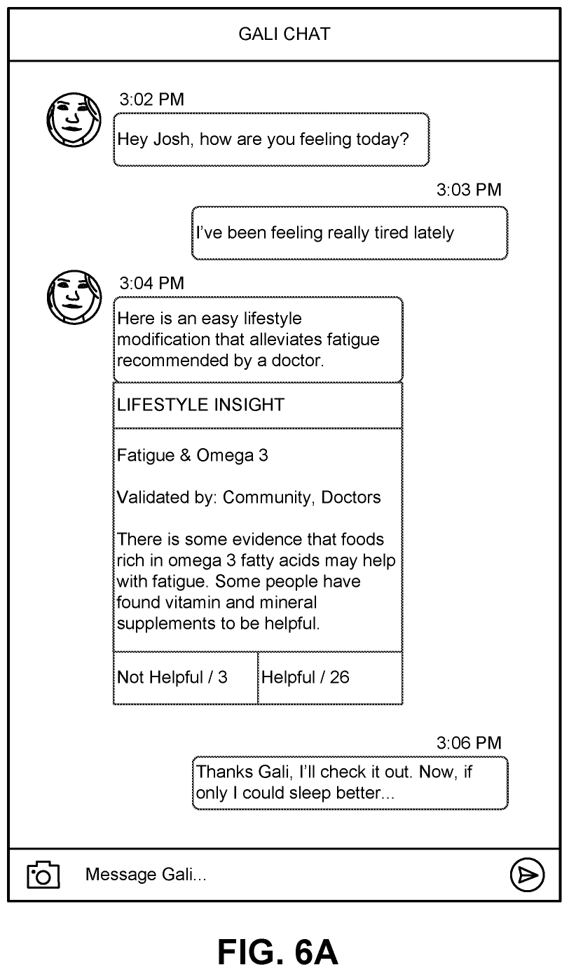

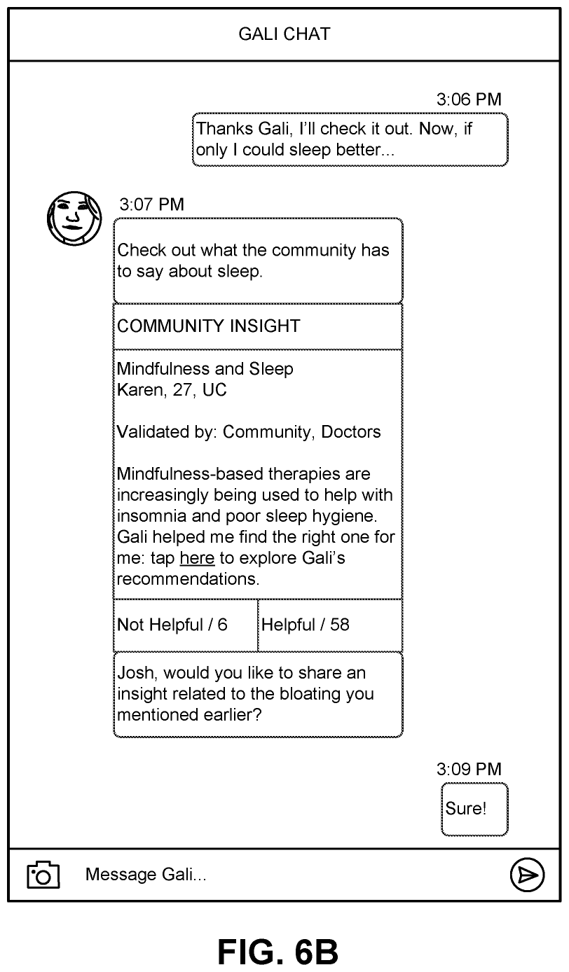

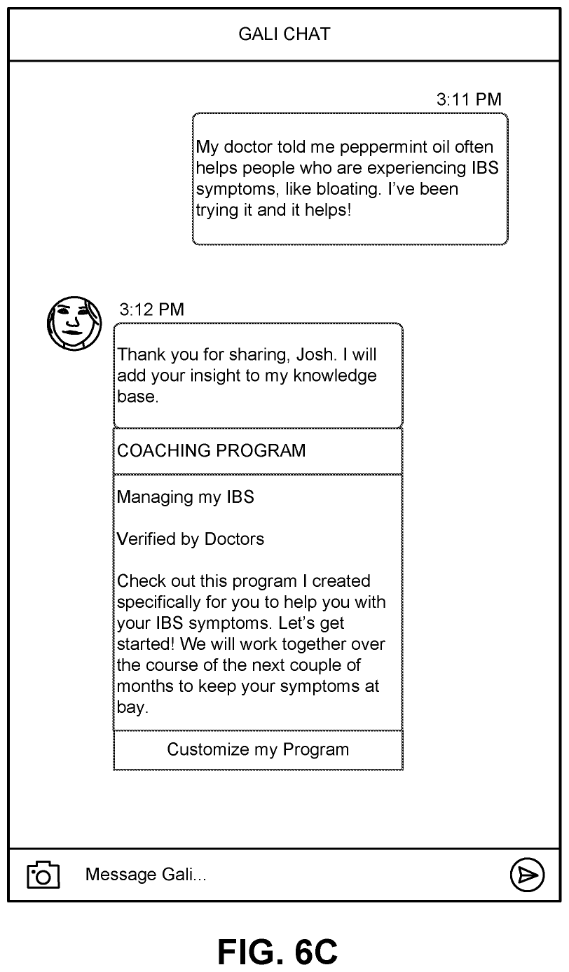

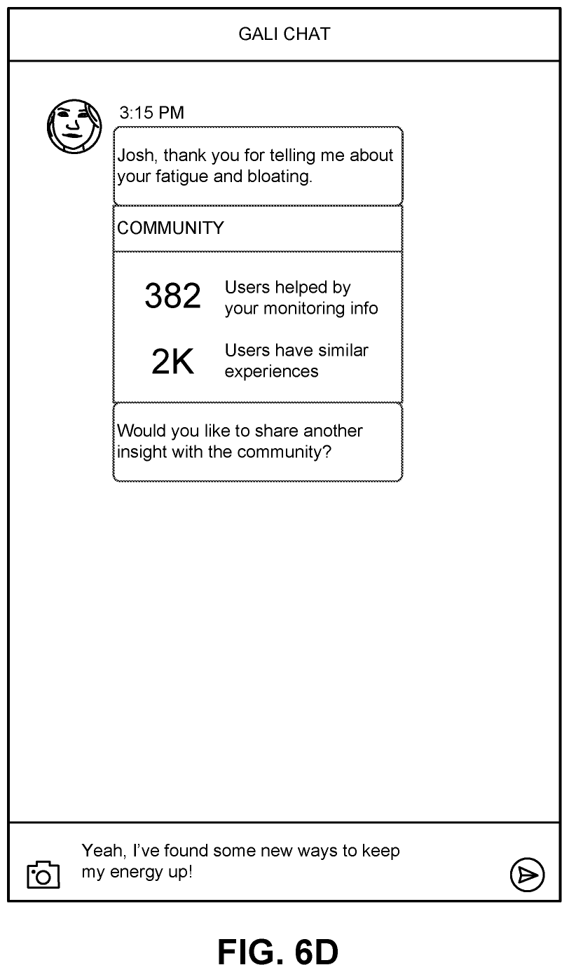

In some embodiments, the interface engine 200 includes a conversation engine that uses artificial intelligence algorithms or machine learning algorithms to provide a chatbot interface. The algorithms may include natural language processing techniques known to one skilled in the art. The chatbot interface can serve as an on-demand digital health assistant that learns and adapts to attributes of specific users. For instance, the digital health assistant provides health insights relevant to a particular disease or condition diagnosed for a user. The digital health assistant may also develop a personality using linguistic or semantic analysis of communication with a user. The digital health assistant may communicate in verbal (e.g., text messages) or audio form (e.g., using text-to-speech algorithms). The interface engine 200 may receive user audio recorded from a microphone of a client device 110. An example session with a digital health assistant is further described with reference to FIGS. 6A-D.

In an embodiment, the interface engine 200 provides an onboarding process for a user joining the digital health system 100. During the onboarding process, the interface engine 200 may provide instructions for the user to provide user information via a user interface of a client device 110, e.g., in a chatbot interface. The user information may include, for example, demographics (e.g., gender, age, or ethnicity), current and historical health data (e.g., symptoms, prior surgeries, treatment, smoking, allergies, diet, or exercise), data describing health information in the user's family (e.g., hereditary conditions or genetics), psychosocial factors, employment, education, relationships, hobbies, etc. The interface engine 200 may receive information describing a present treatment plan of the user, which includes data such as frequency and dosage of medication, current or previous side effects, or notification preferences (e.g., for medication reminders or doctor appointments). Moreover, the interface engine 200 may receive an indication of the user's needs, for example, health education, disease management, connection to a patient community, emotional support, or general health management. The interface engine 200 stores the user information in the user data store 210 (e.g., as a “personal health record”) and may associate the user information with a timestamp or a code, for instance, indicating a type of the information (e.g., symptoms start or stop, appointment, procedure, or treatment start of stop).

The interface engine 200 may provide personalized questions to request information regarding a particular type of health condition, e.g., during an onboarding process with a digital health assistant. The questions may be customized based on context determined from previously questions answered by a user. The digital health assistant may personalize questions based on output of one or more machine learning models, which are further described below. In an example use case to diagnose a tumor, the digital health assistant may present questions to a user such as “did you find a mass?”, “what was the result of your mammogram?”, “have you done a breast ultrasound?”, “have you done a breast MRI?”, “what is your estrogen receptor status?”, or “do you take any major medications?” For a given question, the digital health assistant may provide a list of responses from which the user may select, or may allow the user to input text. As a different example, responsive to determining that user is diagnosed with Crohn's granulomatous colitis, the digital health assistant may follow up with subsequent questions regarding whether the user has experienced liquid or soft stool, constipation, or abdominal pain. The digital health assistant may also request information related to a health timeline of the user, e.g., describing an initial time of diagnosis of a condition or disease, when the user last had a major procedure, or when an upcoming procedure is scheduled.

In an example onboarding process or session, the digital health assistant may receive a selection from a user to update treatment information. Responsive to the selection, the digital health assistant presents a user interface with user inputs (e.g., graphical buttons) for “treatment” (e.g., dosage, quantity, or unit), “schedule” (e.g., number of times a day to take the treatment, start date, end date, count, or refill), and “details” (e.g., provider, first prescribed date, pharmacy, or other notes) of a new treatment. Based on the user's inputs, the digital health assistant may provide feedback via another component of the digital health system 100, e.g., the monitor engine 240, support engine 250, or goal engine 260. Further, the digital health assistant may present a graphical user interface showing a timeline of a temporal model including the updated treatment information. The user may view information about physiological events on the user's timeline and learn about various aspects of the user's health such as ways to prevent unwanted side effects of medical treatments. The digital health assistant may provide advice regarding precautions before taking a medical treatment (e.g., Mesalamine) such as informing a doctor and pharmacist about other medications or products that the user is currently taking or plans to take. In addition to updating treatment information, users may also select to add or update information about a procedure (e.g., a colonoscopy, upper endoscopy, or capsule endoscopy) using the options presented by the digital health assistant.

Following an onboarding process, the interface engine 200 may continue to receive information from the user and update the corresponding user information stored in the user data store 210. For example, the user visits a doctor who prescribes a new medication. The user may provide this information and any side effects experienced as result of the new medication, in order to keep up-to-date the information of the digital health system 100. The interface engine 200 may also present questions to a user to monitor the user's health on a periodic basis or responsive to a specific action or event, e.g., taking medication. The interface engine 200 may receive ratings or metrics input by users responding to questions. For example, a rating indicates how well the user feels in general, how well the user slept last night, a current level of stress, or the user's level of physical activity, among other types of health related metrics. In some embodiments, the interface engine 200 may generate anonymized data based on user information of a population of users, which may be organized based on parameters such as age range, geographic region, ethnicity, gender, socioeconomic factors, etc.

Additionally, the interface engine 200 may receive health data from providers 120 and store the health data in the health data store 215 to build a knowledgebase (KB). The knowledgebase includes, for example, formal medical knowledge (e.g., published guidelines, research, or literature), data driven evidence (e.g., drug effectiveness deduced from clinical trials), and community aggregate data (e.g., population statistics and risk factors). In some embodiments, the knowledgebase is curated by health professionals.

The event classifier 220 identifies and classifies physiological events of a user based on information from the user data store 210 and health data store 215. The event classifier 220 may classify the events based on the source of the information (e.g., from a user during an onboarding process or a certain type of provider 120), or a code associated with the information. Classifications may include psychosocial events (e.g., positive or negative emotions, pro- or anti-social behavior, or a level of knowledge) as well as medical events (e.g., symptoms, diagnoses, appointments, treatments, procedures, or non-compliance). In some embodiments, the event classifier 220 processes information received by the digital health system 100 (e.g., during run time) that is not necessarily stored in the user data store 210 and health data store 215.

In an embodiment, the event classifier 220 uses one or more of the following types of medical codes: symptoms start or stop, appointment, procedure, diagnosis, treatment start or stop, and non-compliance start or stop. In addition to classification of medical codes, the event classifier 220 may also determine a psychosocial code for an experience or event of a user. Example psychosocial codes may correspond to: knowledge of symptoms, knowledge of a procedure, knowledge of a disease, fear or shame of negative symptoms, antisocial with friends or at a job, anxiety of a procedure, denial management, relief associated with positive symptoms, prosocial or antisocial romance, or social support. The digital health system 100 may collect information describing psychosocial experiences associated with a medical experience. For instance, a user begins to feel sick, which is classified under the “symptoms start” medical code. The user's psychosocial experiences associated with the start of the symptoms may include confusion as to why the user is having the symptoms (e.g., psychosocial code “knowledge of symptoms”), becoming frightened that something is wrong upon seeing blood in the user's stool (e.g., psychosocial code “fear of negative symptoms”), or being embarrassed to ask for help from friends (e.g., psychosocial codes “antisocial with friends” and “shame of negative symptoms”).

In some embodiments, the event classifier 220 may also determine monitor codes or support codes for recommendations provided by a digital health assistant to help treat a user's disease or condition. Example monitor codes include physical workup, psychological workup, pharmacy medications, symptoms, and side effects. Example support codes include encouragement regarding a disease or symptoms, education about disease, medication management, community treatment, pharmacy reminder, relief from shame, and prosocial encouragement. Responsive to determining that a user is diagnosed with a disease, the digital health assistant may provide background information about the disease (e.g., associated with monitor code “psychological workup” and support code “education about disease”) and recommend that the user take medication (e.g., associated with monitor code “pharmacy medications” and support code “medication management”).

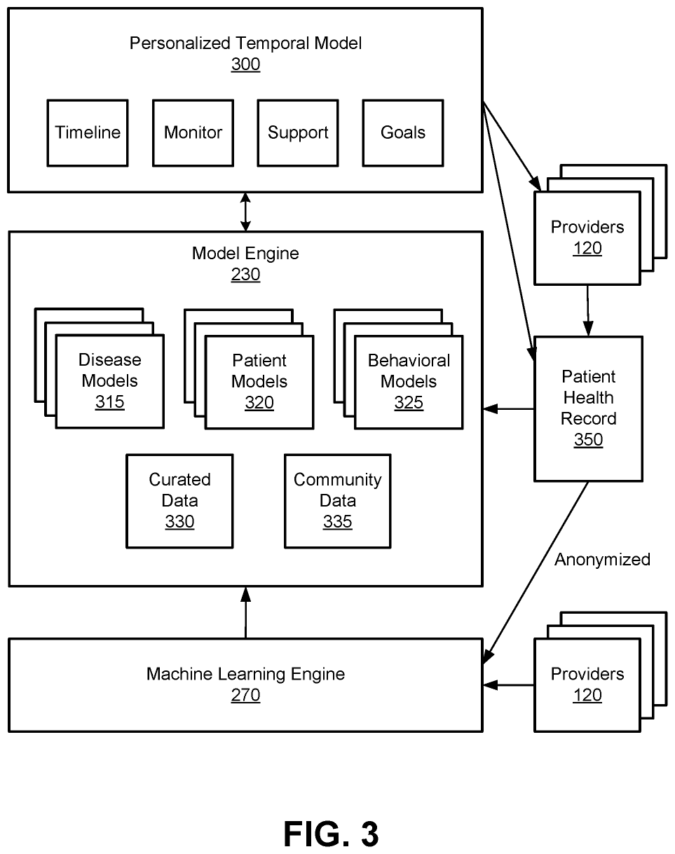

The model engine 230 generates models that analyze user information and health data to provide personalized health feedback to users. In an embodiment, the model engine 230 generates patient models that are personalized for users. A patient model may map the user's physiological events (classified by the event classifier 220) to a timeline of the patient's health journey based on the corresponding timestamps. Additionally, the model engine 230 may also generate disease models or behavior models that are used in conjunction with patient models, in some embodiments. The models are described in more detail below with respect to FIG. 3.

The monitor engine 240 continuously (or on a certain periodic basis) tracks the physiological events of a user's patient model to determine an updated physiological state of the user. In particular, the monitor engine 240 determine workups, side effects, symptoms, pharmacy visits, appointments, and procedures, which may be associated with one or more particular events. For example, the monitor engine 240 determines that the user is experiencing a side effect as result of taking a medication over a given time period.

The support engine 250 determines feedback to support users based on one or more of the models or monitored physiological events. Thus, the support engine 250 may personalize the feedback such that it is a relevant response to the user's specific health information and timeline of events. The feedback may include, for example, reminders for an appointment or to pickup medication at a pharmacy, educational material describing a user's condition or symptoms, encouragement, relief for anti-social behavior, or feedback from a community of a user's family and friends.

The goal engine 260 determines personalized goals for users based on one or more of the models or monitored physiological events. The goals may be associated with tasks that educate a user, monitor the user's health, or encourage the user to make progress in managing any diseases. By providing custom goals that have measurable outcomes, the goal engine 260 may increase the likelihood that the user (or the user's caretakers) will become and stay engaged in learning and improving the user's health conditions or behavior.

In some embodiments, the goal engine 260 determines goals based on a score, which may also be referred to as a “disease management score.” Since various factors contribute to a user's health and methods of improving health, the goal engine 260 may compute the score using parameters associated with the user's education, lifestyle, health, medication, social aspects, health providers (e.g., doctors, nurses, or caretakers), or other custom factors. For instance, a user's score will improve responsive to the user becoming educated about the user's conditions and symptoms, or responsive to the user inputting health data to the digital health system 100 on a regular basis (e.g., at least a threshold number of times within a predetermined period of time). Different types of goals may be related to the aforementioned factors and include, for example, consuming educational material, updating health data (e.g., symptoms, stress levels, nutrition, exercise, social activity, etc.), adhering to a treatment or program, attending scheduled appointments and tests, or custom goals such as improving mobility or stopping medications that reduce quality of life.

The machine learning engine 270 uses machine learning techniques to train one or more of the models generated by the model engine 230. The machine learning engine 270 trains models based on feature vectors derived from historical actions performed by users of the digital health system 100. To generate feature vectors, the machine learning engine 270 may retrieve information from other components of the digital health system 100 such as the user data store 210 and the health data store 215. The machine learning engine 270 may implement machine learning techniques such as deep learning, logistic regression, convolutional neural networks, or other types of dimensionality reduction processes.

In some embodiments, the machine learning engine 270 labels feature vectors based on characteristics of actions and associated health trajectories of users. A “negative” label may indicate that the feature vector includes information describing a user that developed a health condition or experienced an increase in a level of severity of a health condition. On the other hand, a “positive” label may indicate that the feature vector includes information describing a user that did not develop a health condition or experienced an improvement or recovery from a health condition. Thus, a model can learn the types of actions, physiological events, or other health information related to user that is likely to increase or decrease the probability that the user will experience a particular health trajectory. As the digital health system 100 receives additional user and health information, the machine learning engine 270 may re-train or update existing models.

FIG. 3 is a data flow diagram of the digital health system 100 according to one embodiment. As previously described, the model engine 230 may generate disease models 315, patient models 320, and/or behavioral models 325. A disease model 315 is a framework that includes various health trajectories for a particular health condition or disease. Given a user's diagnosis, health background, or preferences, the disease model 315 may determine a health trajectory of the user, as well as feedback for treatment, care, or lifestyle choices to improve the user's quality of life. Features used to train disease models 315 may include, for example, diagnostic classes, high-level summaries of key interventions (e.g., procedures or treatments) associated with a trajectory, milestones or phases of a disease treatment trajectory (e.g., workup, surgery, therapy, surveillance, etc.), disease rankings, disease recurrence of survival metrics, or disease timelines. As an example, the disease model 315 may take into account an estimated timeline of physiological events, e.g., a disease that was diagnosed several months ago is treated differently than a disease that was diagnosed within the past week.

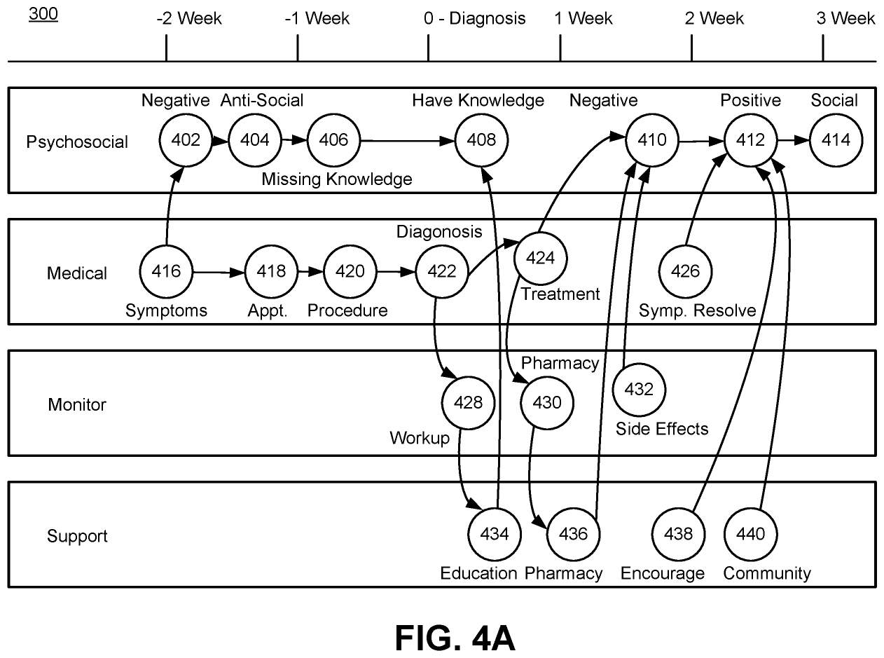

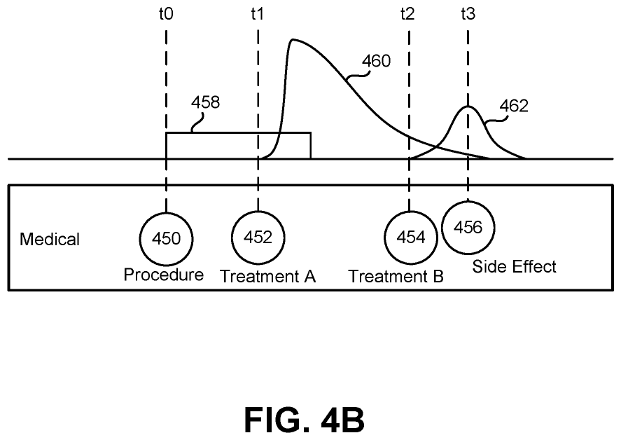

The patient models 320 may include general models based on aggregate data of a population of users and personalized temporal models 300 (also referred to herein as “temporal models”) for particular users. A personalized temporal model 300 includes a timeline of physiological events of a given user. Further, the monitor engine 240, support engine 250, and goal engine 260 may use the personalized temporal model 300 to provide monitoring, support, and goals for the user's health, respectively. In addition to providing feedback to the user, the personalized temporal model 300 may also provide feedback or other information to providers 120 or to update a patent health record 350 (e.g., stored by a third party or in the user data store 210). In some embodiments, a user interacts with the patient models 320 via the previously described digital health assistant, which provides feedback based on output of the models responsive to the user's input (e.g., a question for information about a certain disease and associated symptoms). A more detailed example of a personalized temporal model 300 is described with respect to FIG. 4A.

In some embodiments, the model engine 230 combines information from the disease models 315 and patient models 320, along with behavioral algorithms, to generate the behavioral models 325. The behavioral models 325 may define behavioral parameters such as response timing or duration, types of monitoring or responses, type of feedback, frequency of interaction, or other types of conditional parameters. Moreover, the behavioral models 325 may define functions for certain behavioral tasks that provide behavioral logic based on different combinations of inputs. For example, a function for monitoring symptoms may indicate that, responsive to a user inputting a new treatment, the behavioral models 325 will set up remote reminders to monitor the patient's condition for potential side effects.

The model engine 230 uses curated data 330 or community data 335 to generate models, in some embodiments. The curated data 330 may be retrieved from the knowledge of the health data store 215. The community data 335 may include aggregate data for a population of users from patient health records 350 that have been anonymized. In an embodiment, the machine learning engine 270 uses the curated data 330, community data 335, or other information from providers 120 to train the models. By training with aggregate data of users, the models may formalize the users' experiences to derive insights into distinct correlations based on statistics of the data, instead of medical myths or unsubstantiated recommendations such as anecdotal findings that have not been corroborated with evidence such as clinical studies or published research.

In some embodiments the model engine 230 validates information for training models or developing the knowledgebase of the digital health system 100. The validation may be based on votes or ratings provided by the community, e.g., users of the digital health system 100. For instance, the digital health system 100 presents a health insight to a user, who may vote “up” or “down” to indicate whether the user agrees with the health insight or finds the health insight helpful. The model engine 230 may weigh input from certain users more heavily than input from other users, e.g., a health professional's opinion is applied a greater weight than input form a typical user of the digital health system 100.

### III. Example Personalized Temporal Model

FIG. 4A is a diagram of the personalized temporal model 300 for a user of the digital health system 100 according to one embodiment. In the example shown in FIG. 4A, the personalized temporal model 300 includes a timeline of physiological events from two weeks before a diagnosis of the user to three weeks after the diagnosis. Additionally, the timeline shows events in four categories: psychosocial, medical, monitor, and support. The monitor and support events may be determined by the monitor engine 240 and support engine 250, respectively. The personalized temporal model 300 may also use any combination of disease, patient, or behavioral models to determine different categories of events. In the medical category, the user experiences symptoms 416 two weeks before the diagnosis, followed by an appointment 418 with a doctor (or another health provider) to evaluate the symptoms, and undergoes a procedure 420 to determine a diagnosis. After the user receives a diagnosis 422 of a potential physiological condition (e.g., a disease), the user receives a corresponding treatment 424 about a week after the diagnosis 422, and the symptoms resolve 426 about two weeks after the diagnosis 422. The digital health system 100 may develop the personalized temporal model 300 using information from the user's interactions with a digital health assistant. Furthermore, the digital health assistant may customize health insights or sessions with the user based on knowledge learned from the personalized temporal model 300.

The digital health system 100 may associate events in different categories of the timeline with each other, as illustrated by the arrows shown in FIG. 4A. In the embodiment shown in FIG. 4A, in response to the symptoms 416, the user may experience negative emotions 402, e.g., worrying about a possible disease the user may have developed. Due to these negative emotions, the user may become anti-social 404 or experience other types of mental health conditions such as depression or anxiety. After the appointment 418, the user may be missing knowledge 406 because the diagnosis 422 has not yet been determined. Following the diagnosis 422, the user undergoes a medical workup 428 (e.g., including a physical exam, laboratory tests, x-rays, etc.) and education 434 to learn about the diagnosed physiological condition, resulting in the user having knowledge 408 about their health. Thus, the monitor and support category events may help improve the user's psychosocial state.

Following the treatment 424, the user goes to the pharmacy 430 for medication, e.g., to receive drugs such as antibiotics for post-surgery pain. If the initial medication is not effective, the user may make another visit to the pharmacy 436 to receive additional medication. The user experiences side effects 432 from taking the medications, which causes the user to experience more negation emotions 410. The user receives encouragement 438 from the digital health system 100 and support from the user's community 440 to help improve the user's psychosocial condition. These events, in addition to the symptoms resolving 426 about two weeks after the diagnosis contribute to the user changing a positive psychosocial state 412. With the symptoms resolved and in a better mood, the user participates in a social event 414 about three weeks after the diagnosis.

As illustrated by the timeline of psychosocial, medical, monitor, and support events of the user, the digital health system 100 codifies the user's experiences to determine and provide user-specific feedback and health management on an ongoing basis. By regularly maintaining updated health data on the user, the digital health system 100 enables users to play an active role in making health choices. Additionally, the digital health system 100 enables health providers to better monitor and evaluate the user's condition over time and to make joint decisions with users that are based on empirical data or statistics.

FIG. 4B is a diagram of a timeline of the personalized temporal model 300 illustrating statistical distributions according to one embodiment. As shown in FIG. 4B, the user undergoes a procedure 450 at timestamp t0, starts taking treatment A 452 and treatment B 454 at t1 and t2, respectively, and experiences a side effect 456 at t3. In an example use case, the procedure 450 is a surgery for a user diagnosed with inflammatory bowel disease (IBD) or breast cancer. A physician may prescribe a treatment A 452 such as antibiotics for the user to take within a window of time 458 following the procedure 450. Additionally, the user make take another treatment B 454 such as painkillers. The personalized temporal model 300 may determine the potency of the medical treatments based on statistical distributions of values. In particular, the statistical distributions 460 and 462 represent the effects of treatment A 452 and treatment B 454 on the user's body, respectively (e.g., based on changes in dosage of the medication or chemical reactions in the user's body after consumption). Accordingly, the potency of the antibiotic and painkiller treatments may increase shortly after the user initially takes the medication and then decrease as the effect wears off over time.

In some embodiments, a user may experience a side effect responsive to an overlapping range of two or more statistical distributions, e.g., due to interactions between different medications (e.g., an adverse chemical or physiological reaction), therapies, or other types of treatment. For example, as illustrated in FIG. 4B, a portion of the statistical distributions 460 and 462 overlap starting at t2. The side effect 456 may occur when the potency of the treatment B 454 increases above a threshold value, e.g., at timestamp t3 when the statistical distribution 462 reaches a peak value. Though the above mentioned example relates to medical treatments and side effects, the personalized temporal model 300 may also determine physiological conditions related to emotional or psychological health, in addition to physical health. Furthermore, some treatments or physiological events may be associated with a statistical distribution that is binary (e.g., contagious or not for a disease) or categorical (e.g., low, medium, or high severity of symptoms) instead of a range of values as shown in FIG. 4B.

### IV. Example Process Flow

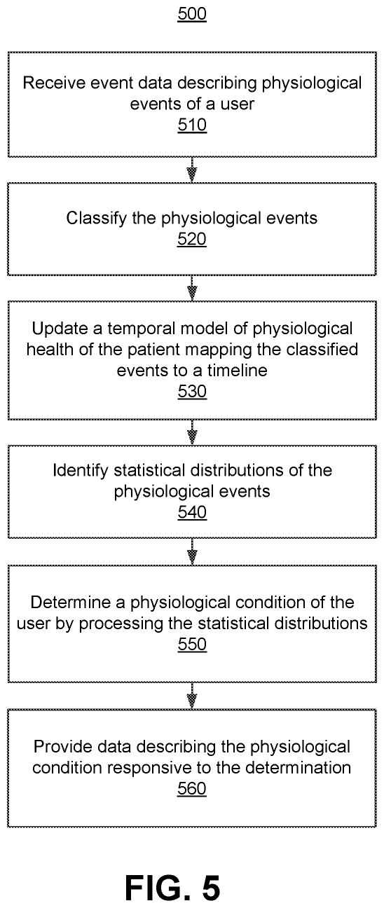

FIG. 5 is a flow chart illustrating a process 500 for providing data describing a physiological condition according to one embodiment. The process 500 may include different or additional steps than those described in conjunction with FIG. 5 in some embodiments, or perform steps in different orders than the order described in conjunction with FIG. 5.

In one embodiment, the interface engine 200 receives 510 event data received from at least a client device 110 of a user. The event data describes physiological events of a user, and each event may have a timestamp. The event classifier 220 classifies 520 each of the physiological events. The model engine 230 updates 530 a temporal model of physiological health of the user using the classified physiological events. In particular, the temporal model maps the classified physiological events to a timeline based on the corresponding timestamps. The temporal model identifies 540 statistical distributions of the physiological events, where the statistical distributions each map a range of values over a portion of the timeline. The temporal model determines 550 a physiological condition of the user by processing values in at least an overlapping range of the statistical distributions. Responsive to the determination of the physiological condition, the digital health system 100 (e.g., the monitor engine 240, support engine 250, or goal engine 260) provides 560 data describing the physiological condition to the client device 110 for presentation to the user. The data may be presented as a summary report or a regular health state report describing the user's health trajectories, and the digital health system 100 may provide the reports to a healthcare provider of the user (e.g., physician, nurse, caretaker, etc.).

In some embodiments, the event classifier 220 classifies a first physiological event as a symptom of a disease (e.g., along with a level of severity), a second physiological event as a diagnosis of the disease, and a third physiological event as a treatment for the disease. Responsive to determining that the physiological condition is a side effect of the treatment, the monitor engine 240 may provide a recommendation for the user to visit a health provider to address the side effect. The temporal model may also determine a range of possibilities, mapped to the timeline, that the user will experience a recurrence of the symptom of the disease. In another example, responsive to identifying a physiological event indicating that the user is exhibiting anti-social behavior or negative emotions, the support engine 250 may provide a recommendation for the user to participate in social activity.

### V. Example Digital Health Assistant

FIGS. 6A, 6B, 6C, and 6D are diagrams of a user interface for a digital health assistant according to various embodiments. The digital health system 100 may provide health insights, recommendations, feedback, or other health-related information data to users via a chat interface of the digital health assistant. The digital health assistant may request and receive input from users via the chat interface. Additionally, the digital health assistant may determine chat responses, recommendations, or health insights based on the previously described trained models or outputs of other components of the digital health system 100.

In the example user interface of FIG. 6A, the digital health assistant is interacting with a user via a text-based chat. Responsive to the digital health assistant asking how the user is feeling, the user inputs a message indicating that the user has been feeling tired. By detecting that the user is fatigued based on contents of the message, the digital health assistant provides a lifestyle insight that may help alleviate the user's fatigue. The lifestyle insight may be recommended by a doctor and validated by users in the community. In some embodiments, the digital health assistant selects recommendations or insights responsive to determining that the recommendations or insights have at least a threshold reputation. For instance, the threshold reputation is based on a threshold number of positive feedback from users such as an “up” vote or a “helpful” rating. The threshold reputation may also be based on a reputation of the doctor.

In the example user interface of FIG. 6B, responsive to determining that the user is also seeking to improve the user's sleep quality, the digital health assistant provides a community insight related to mindfulness and sleep that has been validated by both the users of the community and doctors. Based on the user's health record, timeline of a temporal model, or past interactions with the digital health system 100, the digital health assistant may recall that the user previously mentioned experiencing symptoms of bloating. Thus, the digital health assistant prompts the user to request additional information regarding the bloating.

In the example user interface of FIG. 6C, the user responds to the request of the digital health assistant by providing feedback describing advice from the user's doctor regarding treatment of irritable bowel syndrome (IBS) such as bloating. The digital health assistant adds the user's insight to the knowledgebase of the digital health system 100. The insight may be used to train one or more of the models of the digital health system 100. The digital health system 100 may also recommend a coaching program for managing the user's IBS symptoms. Additionally, the digital health system 100 can customize the program according to information specific to the user that has been collected by the digital health system 100, e.g., through chats, providers, or wearable devices.

In the example user interface of FIG. 6D, the digital health assistant provides a message thanking the user for providing information regarding the user's fatigue and bloating. To encourage the user to share additional information to the digital health system 100, the digital health assistant presents metrics indicating a number of users that were helped using the user's monitoring information and another number of users that have similar experiences.

### VI. Alternative Embodiments

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable non-transitory medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method comprising:
receiving event data describing a plurality of physiological events of a user, each having a timestamp, the event data received from at least a client device of a user;
classifying each of the plurality of physiological events;
updating a temporal model of physiological health of the user using the classified physiological events, the temporal model mapping the classified physiological events to a timeline based on the corresponding timestamps;
identifying statistical distributions of the plurality of the physiological events, the statistical distributions each mapping a range of values over a portion of the timeline;
determining a physiological condition of the user by processing values in at least an overlapping range of the statistical distributions; and
responsive to the determination, providing data describing the physiological condition to the client device for presentation to the user.

2. The method of claim 1, further comprising:
determining a goal for the user based on the physiological condition;
mapping the goal to the timeline; and
providing the goal to the client device for presentation to the user.

3. The method of claim 1, wherein classifying each of the plurality of physiological events further comprises:
determining that a first of the physiological events is a symptom of a disease, and wherein the corresponding range of values indicates severity of the disease over the corresponding portion of the timeline;
determining that a second of the physiological events is a diagnosis of the disease; and
determining that a third of the physiological events is a medical treatment for the disease, wherein the corresponding range of values indicate potency of the medical treatment over the corresponding portion of the timeline.

4. The method of claim 3, wherein determining the physiological condition further comprises:
determining that the user is experiencing a side effect of the medical treatment based on the corresponding potency, and wherein the data describing the physiological condition includes a recommendation to visit a health provider.

5. The method of claim 3, wherein determining the physiological condition further comprises:
determining a range of probabilities, mapped to the timeline, that the user will experience a recurrence of the symptom of the disease.

6. The method of claim 3, wherein classifying each of the plurality of physiological events further comprises:
determining that a fourth of the physiological events is another medical treatment; and
wherein determining the physiological condition further comprises determining that the user is experiencing a side effect of the other medical treatment in response to determining that the ranges of values of the medical treatment and the other medical treatment overlap at least partially on the timeline.

7. The method of claim 1, wherein classifying each of the plurality of physiological events further comprises:
determining that one of the physiological events is a change in psychological state of the user, and wherein the corresponding range of values indicate levels of negative emotion of the psychological state over the corresponding portion of the timeline; and
determining that a subsequent one of the physiological events is anti-social behavior of the user based on the levels of negative emotion.

8. The method of claim 7, wherein determining the physiological condition further comprises:
determining that the user is experiencing a psychological condition, and wherein the data describing the physiological condition includes a recommendation to participate in social activity.

9. The method of claim 1, wherein the event data is further received from one or more of: a health provider of the user, electronic health records of the user, or a wearable device of the user.

10. The method of claim 1, further comprising:
providing, via a chatbot interface, a request for the event data from the user; and
wherein the data describing the physiological condition provided to the client device is presented via the chatbot interface.

11. A computer program product comprising a non-transitory computer readable storage medium having instructions encoded thereon that, when executed by a processor, cause the processor to:
receive event data describing a plurality of physiological events of a user, each having a timestamp, the event data received from at least a client device of a user;
classify each of the plurality of physiological events;
update a temporal model of physiological health of the user using the classified physiological events, the temporal model mapping the classified physiological events to a timeline based on the corresponding timestamps;
identify statistical distributions of the plurality of the physiological events, the statistical distributions each mapping a range of values over a portion of the timeline;
determine a physiological condition of the user by processing values in at least an overlapping range of the statistical distributions; and
responsive to the determination, providing data describing the physiological condition to the client device for presentation to the user.

12. The non-transitory computer readable storage medium of claim 11, having further instructions that when executed by the processor cause the processor to:
determine a goal for the user based on the physiological condition;
map the goal to the timeline; and
provide the goal to the client device for presentation to the user.

13. The non-transitory computer readable storage medium of claim 11, wherein the processor is further caused, when classifying each of the plurality of physiological events, to:
determine that a first of the physiological events is a symptom of a disease, and wherein the corresponding range of values indicates severity of the disease over the corresponding portion of the timeline;
determine that a second of the physiological events is a diagnosis of the disease; and
determine that a third of the physiological events is a medical treatment for the disease, wherein the corresponding range of values indicate potency of the medical treatment over the corresponding portion of the timeline.

14. The non-transitory computer readable storage medium of claim 13, wherein the processor is further caused, when determining the physiological condition, to:
determine that the user is experiencing a side effect of the medical treatment based on the corresponding potency, and wherein the data describing the physiological condition includes a recommendation to visit a health provider.

15. The non-transitory computer readable storage medium of claim 13, wherein the processor is further caused, when determining the physiological condition, to:
determine a range of probabilities, mapped to the timeline, that the user will experience a recurrence of the symptom of the disease.

16. The non-transitory computer readable storage medium of claim 13, wherein the processor is further caused, when classifying each of the plurality of physiological events, to:
determine that a fourth of the physiological events is another medical treatment; and
wherein determining the physiological condition further comprises determining that the user is experiencing a side effect of the other medical treatment in response to determining that the ranges of values of the medical treatment and the other medical treatment overlap at least partially on the timeline.

17. The non-transitory computer readable storage medium of claim 11, wherein the processor is further caused, when classifying each of the plurality of physiological events, to:
determine that one of the physiological events is a change in psychological state of the user, and wherein the corresponding range of values indicate levels of negative emotion of the psychological state over the corresponding portion of the timeline; and
determine that a subsequent one of the physiological events is anti-social behavior of the user based on the levels of negative emotion.

18. The non-transitory computer readable storage medium of claim 17, wherein the processor is further caused, when determining the physiological condition, to:
determine that the user is experiencing a psychological condition, and wherein the data describing the physiological condition includes a recommendation to participate in social activity.

19. The non-transitory computer readable storage medium of claim 11, wherein the event data is further received from one or more of: a health provider of the user, electronic health records of the user, or a wearable device of the user.

20. The non-transitory computer readable storage medium of claim 11, having further instructions that when executed by the processor cause the processor to:
provide, via a chatbot interface, a request for the event data from the user;
wherein the data describing the physiological condition provided to the client device is presented via the chatbot interface.
